(19) United States
(12) Patent Application Publication
(10) Pub. No.: US 2008/0103152 A1
(43) Pub. Date: May 1, 2008

(54) Novel Diazabicyclononene Derivative

(75) Inventors: Olivier Bezencon, Riehen (CH); Daniel Bur, Allschwil (CH); Walter Fisciili, Allschwil (CH); Lubos Remen, Allschwil (CH); Sylvia Richard-Bildstein, Dietwiller (FR); Thierry Sifferlen, Guewenheim (FR); Hans-Peter Weber, Hochwald (CH); Thomas Weller, Binningen (CH)

Correspondence Address: Thomas Hoxie, Hoxie & Tso LLP, 374 Millburn Avenue, Suite 300E, Millburn, NJ, 07041, US

(73) Assignee: Actelion Pharmaceuticals Ltd, Allschwil (CH)

(21) Appl. No.: 11/792,728

(22) PCT Filed: Dec. 8, 2005
(86) PCT No.: PCT/IB05/54113

§ 371 Date: Jun. 7, 2007

(30) Foreign Application Priority Data
Dec. 8, 2004 (EP) PCT/EP2004/013949

(51) Int. Cl. A61K 31/4995; A61P 13/12; A61P 43/00; A61P 7/12; C07D 471/08

(52) U.S. Cl. 514/249; 544/349

(57) ABSTRACT

The invention relates to a novel 3,9-diazabicyclo[3.3.1]nonene derivative and the enantiomers thereof and the use thereof as active ingredients in the preparation of pharmaceutical compositions. The invention also concerns related aspects including processes for the preparation of the compounds, pharmaceutical compositions containing at least one compound of formula (I) or (I′) and especially their use as inhibitors of renin.

The invention relates to novel compounds of the formula (I) and the enatiomer thereof of formula (I′). The invention also concerns related aspects including processes for the preparation of the compounds, pharmaceutical compositions containing at least one compound of formula (I) or (I′) and especially their use as renin inhibitors in cardiovascular events and renal insufficiency.

In the renin-angiotensin system (RAS) the biologically active angiotensin II (Ang II) is generated by a two-step mechanism. The highly specific enzyme renin cleaves angiotensinogen to angiotensin I (Ang I), which is then further processed to Ang II by the less specific angiotensin-converting enzyme (ACE). Ang II is known to work on at least two receptor subtypes called AT₁ and AT₂. Whereas AT₁ seems to transmit most of the known functions of Ang II, the role of AT₂ is still unknown.

Modulation of the RAS represents a major advance in the treatment of cardiovascular diseases. ACE inhibitors and AT₁ blockers have been accepted to treat hypertension (Waeber B. et al., “The renin-angiotensin system: role in experimental and human hypertension”, in Birkenhager W. H., Reid J. L. (eds): Hypertension, Amsterdam, Elsevier Science Publishing Co, 1986, 489-519; Weber M. A., Am. J. Hypertens., 1992, 5, 247S). In addition, ACE inhibitors are used for renal protection (Rosenberg M. E. et al., Kidney International, 1994, 45, 403; Breyer J. A. et al., Kidney International, 1994, 45, S156), in the prevention of congestive heart failure (Vaughan D. E. et al., Cardiovasc. Res., 1994, 28, 159; Fouad-Tarazi F. et al., Am. J. Med., 1988, 84 (Suppl. 3A), 83) and myocardial infarction (Pfeffer M. A. et al., N. Engl. J. Med., 1992, 327, 669).

The rationale to develop renin inhibitors is the specificity of renin (Kleinert H. D., Cardiovasc. Drugs, 1995, 9, 645). The only substrate known for renin is angiotensinogen, which can only be processed (under physiological conditions) by renin. In contrast, ACE can also cleave bradykinin besides Ang I and can be bypassed by chymase, a serine protease (Husain A., J. Hypertens., 1993, 11, 1155). In patients inhibition of ACE thus leads to bradykinin accumulation causing cough (5-20%) and potentially life-threatening angioneurotic edema (0.1-0.2%) (Israili Z. H. et al., Annals of Internal Medicine, 1992, 117, 234). Chymase is not inhibited by ACE inhibitors. Therefore, the formation of Ang II is still possible in patients treated with ACE inhibitors. Blockade of the AT₁ receptor (e.g. by losartan) on the other hand overexposes other AT-receptor subtypes (e.g. AT₂) to Ang II, whose concentration is significantly increased by the blockade of AT₁ receptors. In summary, renin inhibitors are expected to demonstrate a different pharmaceutical profile than ACE inhibitors and AT₁ blockers with regard to efficacy in blocking the RAS and in safety aspects.

Only limited clinical experience (Azizi M. et al., J. Hypertens., 1994, 12, 419; Neutel J. M. et al., Am. Heart, 1991, 122, 1094) has been created with renin inhibitors because of their insufficient oral activity due to their peptidomimetic character (Kleinert H. D., Cardiovasc. Drugs, 1995, 9, 645). The clinical development of several compounds has been stopped because of this problem together with the high cost of goods. Only one compound containing four chiral centers has entered clinical trials (Rahuel J. et al., Chem. Biol., 2000, 7, 493; Mealy N. E., Drugs of the Future, 2001, 26, 1139). Thus, renin inhibitors with good oral bioavailability and long duration of action are required. Recently, the first non-peptide renin inhibitors were described which show high in vitro activity (Oefner C. et al., Chem. Biol., 1999, 6, 127; Patent Application WO 97/09311; Märki H. P. et al., Il Farmaco, 2001, 56, 21). However, the development status of these compounds is not known.

The present invention relates to renin inhibitors of a non-peptidic nature and of low molecular weight. Described are orally active renin inhibitors of formula (I) and (I′) which have a of long duration of action and which are active in indications beyond blood pressure regulation where the tissular renin-chymase system may be activated leading to pathophysiologically altered local functions such as renal, cardiac and vascular remodeling, atherosclerosis, and possibly restenosis.

In particular, the present invention relates to a novel compound of the structural formula (I): (1R*,5S*)-7-{4-[3-(2-chloro-3,6-difluorophenoxy)-propyl]phenyl}-3,9-diazabicyclo[3.3.1]non-6-ene-6-carboxylic acid cyclopropyl-(2,3-dichlorobenzyl)amide

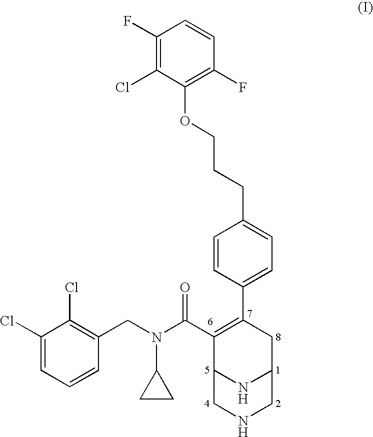

and optically pure enantiomers or a mixture of enantiomers such as a racemate; as well as pharmaceutically acceptable salts, solvent complexes and morphological forms thereof.

A preferred enantiomer is the one represented by formula (I′): (1R,5s)-7-{4-[3-(2-chloro-3,6-difluorophenoxy)propyl]phenyl}-3,9-diazabicyclo[3.3.1]non-6-ene-6-carboxylic acid cyclopropyl-(2,3-dichlorobenzyl)amide

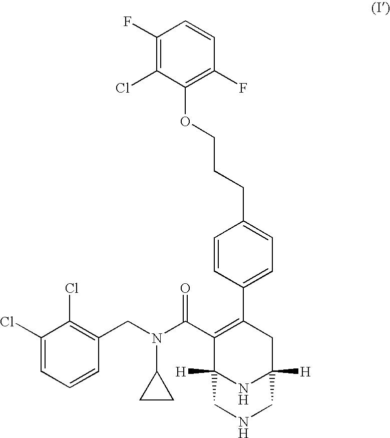

The expression pharmaceutically acceptable salts encompasses either salts with inorganic acids or organic acids like hydrochloric or hydrobromic acid, sulfuric acid, phosphoric acid, citric acid, formic acid, acetic acid, maleic acid, tartaric acid, benzoic acid, methanesulfonic acid, p-toluenesulfonic acid, and the like that are non toxic to living organisms.

The compounds of the formula (I) contain two inter-dependent asymmetric carbon atoms having the relative stereochemistry (1R*,5S*) and may be prepared in form of the optically pure enantiomers (1R,5S)-7-{4-[3-(2-chloro-3,6-difluorophenoxy)-propyl]phenyl}-3,9-diazabicyclo[3.3.1]non-6-ene-6-carboxylic acid cyclopropyl-(2,3-dichlorobenzyl)amide (i.e. compound of formula (I′) which is preferred) and (1S,5R)-7-{4-[3-(2-chloro-3,6-difluorophenoxy)-propyl]phenyl}-3,9-diazabicyclo[3.3.1]non-6-ene-6-carboxylic acid cyclopropyl-(2,3-dichlorobenzyl)amide, or as a mixture of the two enantiomers such as a racemate. The present invention encompasses all these forms. Mixtures may be separated in a manner known per se, i.e. by column chromatography, thin layer chromatography, HPLC, crystallization, or resolution.

Especially a resolution of compound 3 (vide infra) is possible, using tartaric acid. The use of D-(−)-tartaric acid will lead to the desired enantiomer.

The compounds of formula (I) and (I′) are useful for the treatment and/or prophylaxis of diseases associated with a dysregulation of the renin-angiotensin system, in particular diseases such as or related to hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, glomerulonephritis, renal colic, complications resulting from diabetes such as nephropathy, vasculopathy and neuropathy, glaucoma, elevated intra-ocular pressure, atherosclerosis, restenosis post angioplasty, complications following vascular or cardiac surgery, erectile dysfunction, hyperaldosteronism, lung fibrosis, scleroderma, anxiety, cognitive disorders, complications of treatments with immunosuppressive agents, and other diseases known to be related to the renin-angiotensin system.

The compounds of formula (I) and (I′) are especially useful for the treatment and/or prophylaxis of hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, complications resulting from diabetes such as nephropathy, vasculopathy and neuropathy.

In one embodiment, the invention relates to a method for the treatment or prophylaxis of diseases, which are associated with a dysregulation of the renin-angiotensin system, in particular to a method for the treatment or prophylaxis of the above-mentioned diseases, said methods comprising administering to a patient a pharmaceutically active amount of a compound of formula (I) or (I′).

A further aspect of the present invention relates to pharmaceutical compositions comprising a compound of formula (I) or (I′) and a pharmaceutically acceptable carrier material. These pharmaceutical compositions may be used for the treatment and/or prophylaxis of the above-mentioned diseases. The pharmaceutical compositions can be used for enteral, parenteral, or topical administration. They can be administered, for example, perorally, e.g. in the form of tablets, coated tablets, dragées, hard and soft gelatine capsules, solutions, emulsions or suspensions, rectally, e.g. in the form of suppositories, parenterally, e.g. in the form of injection solutions or infusion solutions, or topically, e.g. in the form of ointments, creams or oils.

The invention also relates to the use of a compound of formula (I) or (I′) for the preparation of pharmaceutical compositions for the treatment and/or prophylaxis of the above-mentioned diseases.

The production of the pharmaceutical compositions can be effected in a manner which will be familiar to any person skilled in the art (see for example Mark Gibson, Editor, Pharmaceutical Preformulation and Formulation, IHS Health Group, Englewood, Colo., USA, 2001; Remington, The Science and Practice of Pharmacy, 20th Edition, Philadelphia College of Pharmacy and Science) by bringing the described compounds of formula (I) or (I′) or their pharmaceutically acceptable salts, optionally in combination with other therapeutically valuable substances, into a galenical administration form together with suitable, non-toxic, inert, therapeutically compatible solid or liquid carrier materials and, if desired, usual pharmaceutical adjuvants.

Suitable carrier materials are not only inorganic carrier materials, but also organic carrier materials. Thus, for example, lactose, corn starch or derivatives thereof, talc, stearic acid or its salts can be used as carrier materials for tablets, coated tablets, dragées and hard gelatine capsules. Suitable carrier materials for soft gelatine capsules are, for example, vegetable oils, waxes, fats and semi-solid and liquid polyols (depending on the nature of the active ingredient no carriers are, however, required in the case of soft gelatine capsules). Suitable carrier materials for the production of solutions and syrups are, for example, water, polyols, sucrose, invert sugar and the like. Suitable carrier materials for injections are, for example, water, alcohols, polyols, glycerols and vegetable oils. Suitable carrier materials for suppositories are, for example, natural or hardened oils, waxes, fats and semi-liquid or liquid polyols. Suitable carrier materials for topical preparations are glycerides, semi-synthetic and synthetic glycerides, hydrogenated oils, liquid waxes, liquid paraffins, liquid fatty alcohols, sterols, polyethylene glycols and cellulose derivatives.

Usual stabilizers, preservatives, wetting and emulsifying agents, consistency-improving agents, flavor-improving agents, salts for varying the osmotic pressure, buffer substances, solubilizers, colorants and masking agents and antioxidants come into consideration as pharmaceutical adjuvants.

The dosage of compounds of formula (I) and (I′) can vary within wide limits depending on the disease to be controlled, the age and the individual condition of the patient and the mode of administration, and will, of course, be fitted to the individual requirements in each particular case.

In a preferred embodiment, this amount is comprised between 2 mg and 1000 mg per day.

In a particularly preferred embodiment, this amount is comprised between 1 mg and 500 mg per day.

In a more particularly preferred embodiment, this amount is comprised between 5 mg and 200 mg per day.

Another aspect of the invention is related to a process for the preparation of a pharmaceutical composition comprising a compound of the formula (I) or (I′). According to said process, one or more active ingredients of the formula (I) or (I′) are mixing with inert excipients in a manner known per se.

Compounds of formula (I) and (I′) or the above-mentioned pharmaceutical compositions are also of use in combination with other pharmacologically active compounds such as ACE-inhibitors, neutral endopeptidase inhibitors, angiotensin II receptor antagonists, endothelin receptors antagonists, vasodilators, calcium antagonists, potassium activators, diuretics, sympatholitics, beta-adrenergic antagonists, alpha-adrenergic antagonists, and/or other drugs beneficial for the prevention or the treatment of the above-mentioned diseases such as aldosterone antagonists, 11beta-hydroxysteroid dehydrogenase type 1 inhibitors and soluble guanylate cyclase activators.

The present invention also relates to pro-drugs of a compound of formula (I) or (I′) that convert in vivo to the compound of formula (I) or (I′) as such. Any reference to a compound of formula (I) or (I′) is therefore to be understood as referring also to the corresponding pro-drugs of the compound of formula (I) or (I′), as appropriate and expedient.

The compounds of the formula (I) and (I′) can be manufactured by the methods outlined below, by the method described in the example or by analogous methods.

### Preparation of the Compounds of Formula (I ) and (I′):

The synthesis of the compounds of formula (I) and (I′) described hereby is one possible synthesis among many other alternatives. Other synthetic routes and methodologies will be apparent to the skilled person in the art.

The bicyclononane derivative 3 (Scheme 1) can be prepared as racemate as described earlier (WO 2003/093267). Preparation of the vinylic triflate 6 proceeds using sodium hydride and N-phenyl-bis(trifluoromethanesulfonimide). A Negishi-coupling between the bicyclic system 6 and the bromophenyl derivative 10 leads to diazabicyclonene 7. Bromophenyl derivative 10 is prepared in three steps from 1,4-dibromobenzene, via a Grignard reaction with allyl bromide (→compound 8), then a hydroboration (→compound 9) and finally protection of the hydroxy substituent as a silyl ether. Bicyclononene 7 is then transprotected to bicyclononene 11. A Mitsunobu-type coupling with 2-chloro-3,6-difluorophenol leads to derivative 12. Saponification of the ethyl ester under strongly basic conditions leads to a mixture of carboxylic acid derivatives 13 and 14.

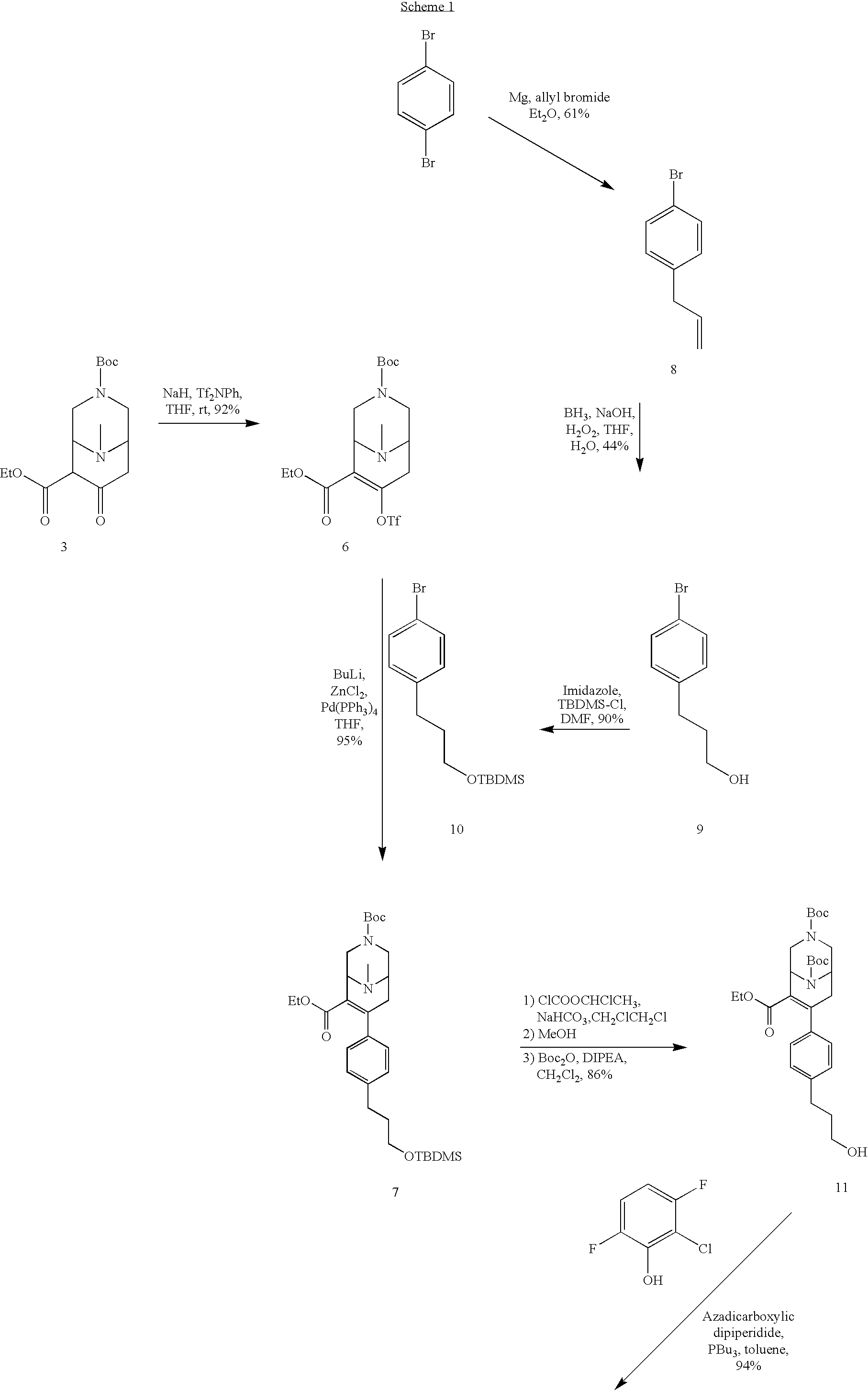

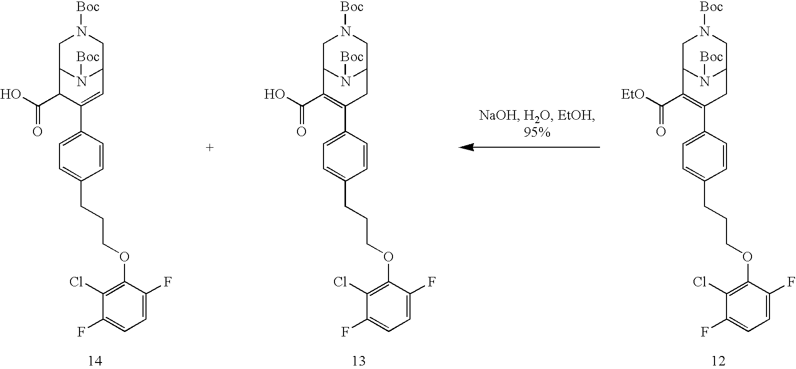

The mixture of compounds 13 and 14 is not separated and used directly further in the amide coupling step with amine derivative 16, which is prepared in one step using a reductive amination (Scheme 2). Amide coupling product 15 is obtained. Cleavage of both Boc-protecting groups leads to a compound of formula (I).

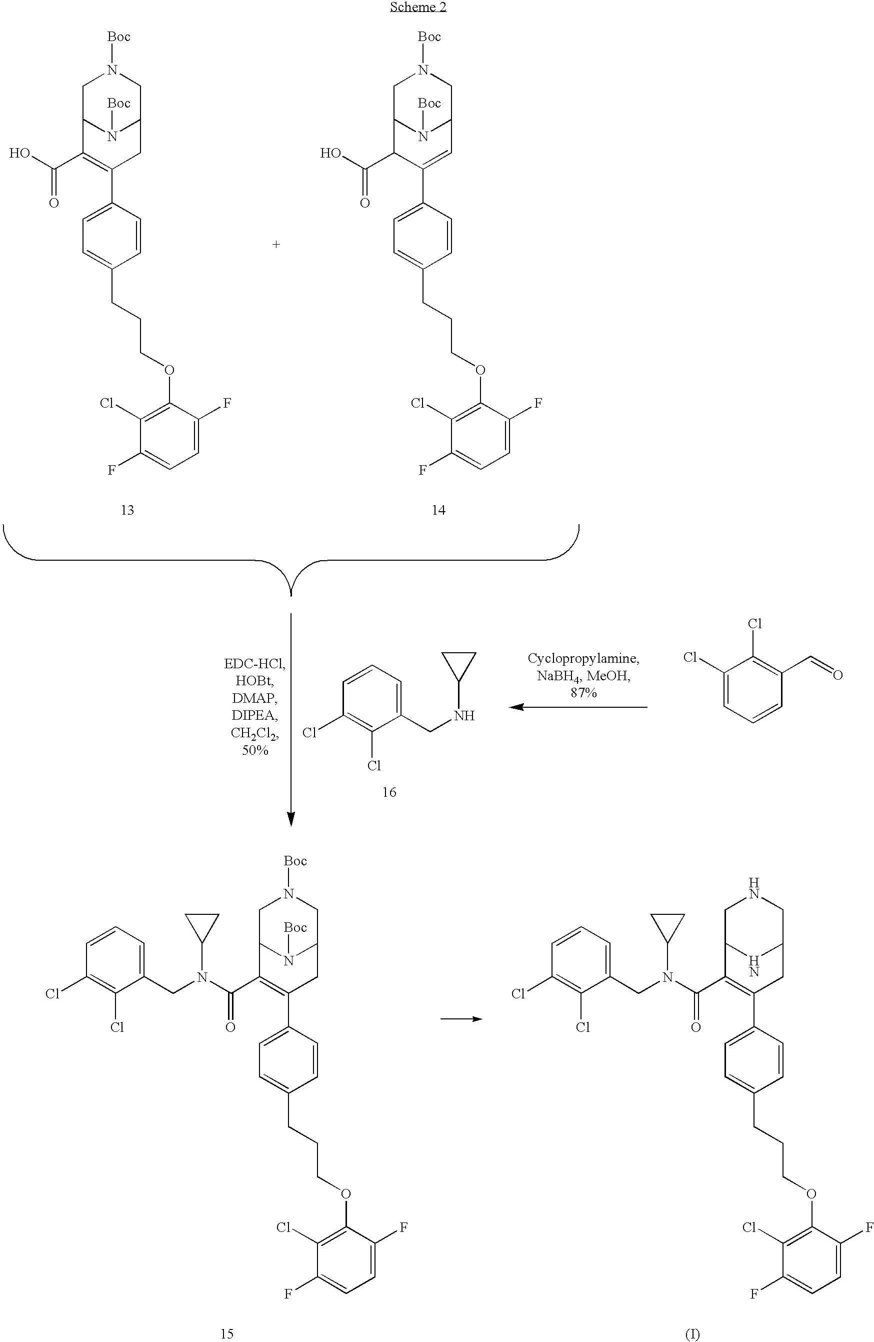

### Enantioselective Synthesis:

The compounds of the present invention contain two chiral centers which, however, are not independent from each other. The synthetic method presented so far leads to a racemate. The racemate can be separated into the compound of formula (I′) and its enantiomer using a chiral HPLC-column. Also, both enantiomers might be prepared selectively starting from a meso-bicyclononane derivative, like compound 17 (Scheme 3), using an enantioselective acylation (Majewski M., Lasny R., J. Org. Chem., 1995, 60, 5825), as described in WO 2003/093267. Another alternative would be a resolution of the racemate using a chiral, organic acid derivative.

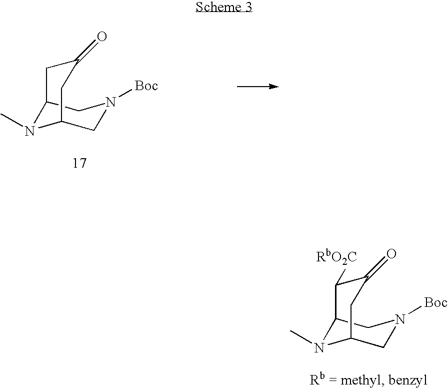

The following examples serve to illustrate the present invention in more detail. It is, however, not intended to limit its scope in any manner.

### EXAMPLES

### General Remarks

The compound is characterized at least by LC-MS and ¹H-NMR. Only the LC-MS data are given here.

HPLC- or LC-MS-conditions (if not indicated otherwise):

Analytic: Zorbax 59 SB Aqua column, 4.6×50 mm from Agilent Technologies. Eluents: A: Acetonitrile; B: H₂O+0.5% TFA. Gradient: 90% B→5% B over 2 min. Flow: 1 mL/min. Detection: UV/Vis+MS.

Preparative: Zorbax SB Aqua column, 20×500 mm from Agilent Technologies. Eluent: A: Acetonitrile; B: H₂O+0.05% ammonium hydroxide (25% aq.). Gradient: 80% B→10% B over 6 min. Flow: 40 mL/min. Detection: UV+MS, or UV+ELSD.

Chiral, analytic: Regis Whelk column, 4.6×250 mm, 10 μm. Eluent A: EtOH+0.05% Et₃N. Eluent B: hexane. Isocratic conditions, 60% B, over 40 min, 1 mL/min. The isocratic mixture may vary, depending on the compounds.

Chiral, preparative: As analytical conditions, but on a Regis Whelk 01 column, 50×250 mm and a flow of 100 mL/min.

All t_{R} are given in min.

### Abbreviations (As Used Herein)

ACE Angiotensin Converting Enzyme
Ang Angiotensin
aq. aqueous
Boc tert-Butyloxycarbonyl
BSA Bovine serum albumine
Bu butyl
BuLi n-Butyllithium
DIPEA Diisopropylethylamine
DMAP 4-N,N-Dimethylaminopyridine
DMF N,N-dimethylformamide
DMSO Dimethylsulfoxide
EDC.HCl Ethyl-N,N-dimethylaminopropylcarbodiimide hydrochloride
EIA Enzyme immunoassay
ELSD Evaporative Light-Scattering Detection
ES+ Electrospray, positive ionization
Et Ethyl
EtOAc Ethyl acetate
EtOH Ethanol
FC Flash Chromatography
h hour(s)
HOBt Hydroxybenzotriazol
HPLC high performance liquid chromatography
LC-MS Liquid chromatography-mass spectrometry
MeOH Methanol
min minute(s)
MS mass spectrometry
org. organic
Ph Phenyl
rt room temperature
sat. saturated
sol. Solution
TBDMS tert-Butyldimethylsilyl
Tf Trifluoromethylsulfonyl
TFA trifluoroacetic acid
THF Tetrahydrofuran
t_{R} retention time
UV ultra violet
vis visible

### Compound 6

Compound 3 (WO 2003/093267, 99.58 g, 305 mmol) was dissolved in dry THF (1450 mL) under a nitrogene atmosphere and the mixture was cooled to 0° C. NaH (16.64 g; 55% dispersion in mineral oil, 381 mmol) was added by portions of 2 g over a period of 35 min, keeping the temperature between 0 and 4° C. H₂ gas evolved. After the addition the mixture was yellow-green and was a slight suspension. The reaction mixture was stirred for 75 min at 0 to 4° C. Tf₂NPh (128.6 g, 360 mmol) was then added as a solid within 5 min. The reaction mixture became brown. The cooling bath was removed and the reaction was stirred over the weekend at rt. The reaction mixture was poured onto 1 L of ice/water and the THF was removed under reduced pressure. The remaining water phase was extracted with (3×500 mL) EtOAc. The combined organic layers were washed with water (500 mL) and brine (500 mL). The organic phase was then dried over MgSO₄, filtered, and evaporated under reduced pressure. To the crude brown residue (174 g) was added 50 mL of pentane and the mixture was stirred at 4° C. overnight. The crystals were filtered off and washed with cold hexane (70 mL) and a cold mixture of hexane/diethylether (4:1, 100 mL). This resulted in 84 g of product containing some TfNHPh. This material was filtered over silicagel (75 g). TfNHPh was washed out with CH₂Cl₂. This product was subsequently washed out with EtOAc (3 times 1 L) to give the title compound after evaporation under reduced pressure. The title compound was obtained in three fractions: a) 44.45 g of off-white crystals, b) 27.98 g of little brown crystals, and c) 15 g of a yellow oil containing the product and TfNHPh. After 2 days the TfNHPh contained in fractions c) had crystallised. It was filtered to yield 9.43 g of the product as a brown oil.

### Treatment of the Mother Liquors:

The combined mother liquors obtained above were concentrated under vacuo. The brown oil residue (75 g) was purified by FC (1500 g silica gel) using a gradient of (EtOAc/heptane 1:9→EtOAc). The column was then washed with EtOAc/MeOH 9:1. The title compound was isolated as 25.44 g of an off-white solid as the pure product. LC-MS: t_{R}=0.87 min; ES+: 459.24.

### Compound 7

A sol. of compound 10 (102.1 g, 310 mmol) in THF (1.50 L) under nitrogen was cooled to −78° C. BuLi (1.5M in hexane, 212 mL, 318 mmol) was added. After 30 min, ZnCl₂ (1M in THF, 465 mL, 465 mmol) was added. The mixture was allowed to warm up to rt. Compound 6 (83.6 g, 155 mmol) in THF (100 mL) and then Pd(PPh₃)₄ (4.48 g, 3.87 mmol) were added. The mixture was heated to reflux for 30 min, and allowed to cool to rt. The mixture was diluted with EtOAc and washed with aq. 1M NaOH (1×). The org. extracts were dried over MgSO₄, filtered and the solvents were removed under reduced pressure. Purification of the residue by FC (MeOH/CH₂Cl₂ 1:49→1:24→3:47→2:23) yielded the title product (88.6 g, virtually quantitative). LC-MS: t_{R}=0.98 min; ES+: 559.24.

### Compound 8

Mg (8.76 g, 360 mmol) was suspended in THF (90 mL) under nitrogen in a three-necked flask equipped with a condenser and a dropping funnel. The dropping funnel was filled with 1,4-dibromobenzene (77.3 g, 327 mmol) in THF (40 mL). About 5% of the 1,4-dibromobenzene-sol. was added carefully to the Mg-suspension and the reaction was started with the help of a heat gun. When the reaction started, the 1,4-dibromobenzene-sol. was added to such a speed so that the reaction mixture was refluxing gently (about 20 min). The mixture was stirred for further 30 min, and was cooled to 0° C. THF (100 mL) was added and the dropping funnel was filled with a sol. of allyl bromide (30.5 mL, 360 mmol) in THF (50 mL). The allyl bromide sol. was added slowly, maintaining the reaction temperature below 20° C. When the addition was complete, the mixture was stirred for further 30 min, while cooling to 0° C. Aq. 1M HCl was added. The mixture was diluted with Et₂O, and washed with aq. 1M HCl and brine. The combined aq. extracts were extracted back with Et₂O. The combined org. extracts were dried over MgSO₄, filtered, and the solvents were removed under reduced pressure. Distillation of the residue (11 mbar, 88-92° C.) yielded the title compound (39.3 g, about 61%), together with another, unidentified impurity.

### Compound 9

BH₃ (1M in THF, 412 mL, 412 mmol) was added to a sol. of compound 8 (204 g, 1.03 mmol) in THF (1.00 L) under nitrogen at 0° C. The mixture was stirred overnight while warming up to rt. Aq. NaOH (2.5M, 1.65 L, 4.12 mol) was added, and the mixture was cooled to 0° C. H₂O₂ (35%, 480 mL, 5.15 mol) was dropped carefully, and the mixture was stirred for 3 h. Et₂O was added, and the phases were separated. The org. layer was washed with water (1×), and brine (1×). The org. layer was dried over MgSO₄, filtered, and the solvents were removed under reduced pressure. Purification by FC (Et₂O/petroleum ether 1:1→Et₂O) yielded the title compound (97.4 g, 44%).

### Compound 10

A sol. of compound 9 (49.4 g, 230 mmol) in DMF (500 mL) was cooled to 0° C., and imidazole (23.77 g, 349 mmol) and TBDMS-Cl (52.6 g, 349 mmol) were added. The mixture was stirred overnight, while warming up to rt. The mixture was diluted with heptane (1.0 L) and aq. sat. NH₄Cl (800 mL), and the mixture was shaken. The layers were separated. The aq. layer was extracted with heptane, and the combined org. extracts were washed with brine. The org. extracts were dried over MgSO₄, filtered, and the solvents were removed under reduced pressure. Purification by FC (Et₂O/heptane 1:99→1:19) yielded the title compound (68.1 g, 90%). LC-MS: t_{R}=1.24 min; ES+: not visible.

### Compound 11

Compound 7 (32.0 g, 57.3 mmol) was dissolved in dry 1,2-dichlorethane (590 mL). NaHCO₃ (48.2 g, 573 mmol) and 1-chloroethyl chloroformate (62.5 mL, 573 mmol) were added, and the suspension was heated to 80° C. After 3 h the reaction mixture was allowed to cool to rt. The mixture was filtered and the filtrate was evaporated under reduced pressure. The residue was dried 15 min under high vacuum. The product was then diluted in MeOH (400 mL), and the mixture was heated to 50° C. for 20 min. The reaction mixture was allowed to cool to rt, and the solvents were removed under reduced pressure. The yellow solid was dried under high vacuum for 1 h. The solid was dissolved in CH₂Cl₂ (190 mL), and the solution was cooled to 0° C. DIPEA (49.1 mL, 287 mmol), and Boc₂O (37.5 g, 172 mmol) were added. The reaction mixture was stirred overnight while warming up to rt. The reaction mixture was diluted with CH₂Cl₂ (110 mL). The organic layer was washed with aq. 1M HCl (2×300 mL), and aq. sat. NaHCO₃ (300 mL). The org. layer was dried over MgSO₄, filtered, and the solvents were evaporated under reduced pressure. Purification of the residue by FC (CH₂Cl₂/MeOH 100:0→2:98→5:95) yielded the title compound (26.2 g, 86%). LC-MS: t_{R}=1.05 min; ES+: 531.33.

### Compound 12

A mixture of compound 11 (56.0 g, 106 mmol), 2-chloro-3,6-difluorophenol (34.8 g, 211 mmol), azadicarboxylic dipiperidide (53.4 g, 211 mmol) and PBu₃ (85%, 83 mL, 317 mmol) in toluene (1.20 L) was heated to reflux under nitrogen for 1 h. The mixture was allowed to cool to rt. The mixture was diluted with EtOAc (2.00 L), and the mixture was washed with aq. 1M NaOH (2×900 mL). The org. extracts were dried over MgSO₄, filtered, and the solvents were removed under reduced pressure. Purification of the residue by FC (EtOAc/heptane 1:19→1:1) yielded the title compound (67.5 g, 94%). LC-MS: t_{R}=1.24 min; ES+: 617.25.

### Compounds 13 and 14

A mixture of compound 12 (67.5 g, 99.6 mmol) in aq. 1M NaOH (700 mL) and EtOH (1.40 L) was stirred at 80° C. overnight. The mixture was partially evaporated under reduced pressure, and EtOAc (500 mL) was added. The aq. phase was acidified with aq. 3M HCl, and the mixture was extracted. The org. layer was separated, dried over MgSO₄, filtered, and the solvents were removed under reduced pressure. The residue was dried under high vacuum, giving a 1:1 mixture of compounds 13 and 14, which was used further without purification (61.8 g, 95%). LC-MS: t_{R}=1.12 and 1.14 min; ES+: 649.37.

### Compound 15

A mixture of compounds 13 and 14 (20.7 g, 32.0 mmol), compound 16 (17.3 g, 80 mmol), EDC.HCl (18.4 g, 96 mmol), HOBt (5.40 g, 40.0 mmol), DMAP (0.98 g, 8.00 mmol) and DIPEA (22.0 mL, 128 mmol) in CH₂Cl₂ (540 mL) was stirred at rt. The reaction was checked each day by LC-MS, and EDC.HCl (6.13 g, 32 mmol) and DIPEA (5.5 mL, 32 mmol) were added if the reaction was not complete. When the reaction was complete, the mixture was diluted with more CH₂Cl₂, and was washed with aq. 1M HCl (3×), and aq. sat. NaHCO₃ (2×). The org. layer was dried over MgSO₄, filtered, and the solvents were removed under reduced pressure. Purification by FC yielded the title compound (16.5 g, 60%). LC-MS: t_{R}=1.29 min; ES+: 848.35.

### Compound 16

A mixture of 2,3-dichlorobenzaldehyde (90.0 g, 514 mmol) and cyclopropylamine (72 mL, 1.02 mol) in dry MeOH (1300 mL) was stirred at rt overnight. The reaction mixture was cooled to 0° C., and NaBH₄ (25.3 g, 668 mmol) was added. The reaction mixture was stiffed again at rt overnight. Ice was added to the reaction mixture, and the solvents were evaporated under reduced pressure. The residue was dissolved in EtOAc, and was washed with aq. 1M NaOH. The aq. layer was extracted with EtOAc (3×). The combined org. extracts were dried over MgSO₄, filtered, and the solvents were removed under reduced pressure. Purification of the residue by FC (EtOAc/heptane 9:1→1:1) yielded the title compound (97.4 g, 87%). LC-MS: t_{R}=0.64 min; ES+: 216.15.

### Compound (I): (rac.)-(1R*,5S*)-7-{4-[3-(2-Chloro-3,6-difluorophenoxy)propyl]phenyl}-3,9-diazabicyclo[3.3.1]non-6-ene-6-carboxylic acid cyclopropyl-(2,3-dichlorobenzyl)amide

A sol. of compound 15 (16.5 g, 19.4 mmol) in CH₂Cl₂ (100 mL) was cooled to 0° C. HCl (4M in dioxane, 100 mL) was added. The mixture was stirred for 1 h at 0° C., then 1 h at rt. The solvents were removed under reduced pressure, and the residue was dried under high vacuum. The residue was diluted with CH₂Cl₂, and washed with aq. 1M NaOH until the aq. layer staid basic. The org. extracts were dried over MgSO₄, filtered, and the solvents were removed under reduced pressure. Purification of the residue by FC (MeOH/CH₂Cl₂ 5:95→10:90→20:80) yielded the title compound (12.0 g, 95%). LC-MS: t_{R}=0.88 min; ES+: 648.36.

### Compound (I′): (1R,5S)-7-{4-[3-(2-Chloro-3,6-difluorophenoxy)propyl]phenyl}-3,9-diazabicyclo[3.3.1]non-6-ene-6-carboxylic acid cyclopropyl-(2,3-dichlorobenzyl)amide

Compound (I) (1.00 g, 1.55 mmol) was purified on a Regis Whelk 01 column, 50×250 mm using isocratic conditions (25% EtOH, 0.1% diethylamine, 75% hexane) and a flow of 100 mL/min. The desired compound came as first enantiomer (t_{R}=20.00 min). After evaporating the solvents under reduced pressure, the residue was dried under high vacuum. The residue was dissolved in CH₂Cl₂, and washed with aq. 10% K₂CO₃. The org. extracts were dried over MgSO₄, filtered, and the solvents were removed under reduced pressure. Drying the residue under high vacuum yielded the title compound (350 mg).

### Biological Assays

### 1. Enzyme Immuno Assay (EIA) to Estimate Ang I Accumulation and Renin Inhibition

### 1.1 Preparation of Ang I-BSA Conjugate

1.3 mg (1 μmol) of Ang I [1-10 (Bachem, H-1680)] and 17 mg (0.26 μmol) of BSA (Fluka, 05475) were dissolved in 4 mL of 0.1M phosphate buffer, pH 7.4, after which 2 mL of a 1:100 dilution of glutaraldehyde in H₂O (Sigma G-5882) was added dropwise. The mixture was incubated overnight at 4° C., then dialyzed against 2 liters of 0.9% NaCl, twice for 4 h at rt, followed by dialysis against 2 liters of PBS 1× overnight at rt. The solution was then filtered with a Syringe filter, 0.45 μm (Nalgene, Cat. No. 194-2545). The conjugate can be stored in polypropylene tubes in 0.05% sodium azide at 4° C. for at least 12 months.

### 1.2 Preparation of BSA-Ang I Coated MTP

Microtiter plates (MPT384, MaxiSorp™, Nunc) were incubated overnight at 4° C. with 80 μl of Ang I (1-10)/BSA conjugate, diluted 1:100,000 in PBS 1× in a teflon beaker (exact dilution dependent on batch of conjugate), emptied, filled with 90 μl of blocking solution [0.5% BSA (Sigma A-2153) in PBS 1×, 0.02% NaN₃], and incubated for at least 2 h at rt, or overnight at 4° C. 96 well MTP (MaxiSorp™, Nunc) were coated with 200 μl conjugate and blocked with 250 μl blocking solution as above, except that the blocking solution contained 3% BSA. The plates can be stored in blocking solution at 4° C. for 1 month.

### 1.3 Ang I-EIA in 384 Well MTP

The Ang I (1-10)/BSA coated MTP were washed 3 times with wash buffer (PBS 1×, 0.01% Tween 20) and filled with 75 μl of primary antibody solution (anti-Ang I antiserum, pre-diluted 1:10 in horse serum), diluted to a final concentration of 1:100,000 in assay buffer (PBS 1×, 1 mM EDTA, 0.1% BSA, pH 7.4). 5 μl of the renin reaction (or standards in assay buffer) (see below) were added to the primary antibody solution and the plates were incubated overnight at 4° C. After the incubation the plates were washed 3 times with wash buffer and incubated with secondary antibody [anti-rabbit IgG, linked to horseradish peroxidase (Amersham Bioscience, NA 934V), diluted 1:2,000 in wash buffer] for 2 h at rt. The plates were washed 3 times with wash buffer and then incubated for 1 h at rt with substrate solution [1.89 mM ABTS (2.2′-azino-di-(3-ethyl-benzthiazolinsulfonate)] (Roche Diagnostics, 102 946) and 2.36 mM H₂O₂ [30%, (Fluka, 95300] in substrate buffer (0.1M sodium acetate, 0.05M sodium dihydrogen phosphate, pH 4.2). The OD of the plate was read at 405 nm in a microplate reader (FLUOStar Optima from BMG). The production of Ang I during the renin reaction was quantified by comparing the OD of the sample with the OD of a standard curve of Ang I (1-10), measured in parallel.

### 2. Primary Renin Inhibition Assay: IC₅₀ in Buffer, 384 Well MTP

The renin assay was adapted from an assay described before (Fischli W. et al., Hypertension, 1991, 18:22-31) and consists of two steps: in the first step, recombinant human renin is incubated with its substrate (commercial human tetradecapeptide renin substrate) to create the product Angiotensin I (Ang I). In the second step, the accumulated Ang I is measured by an immunological assay (enzyme immuno assay, EIA). The detailed description of this assay is found below. The EIA is very sensitive and well suited for renin activity measurements in buffer or in plasma. Due to the low concentration of renin used in this assay (2 fmol per assay tube or 10 pM) it is possible to measure inhibitor affinities in this primary assay down to low pM concentration.

### 2.1 Methodology

Recombinant human renin (3 pg/μl) in assay buffer (PBS 1×, 1 mM EDTA, 0.1% BSA, pH 7.4), human tetradecapeptide (1-14) substrate (Bachem, M-1120) [5 μM in 10 mM HCl], hydroxyquinoline sulfate (Fluka, 55100) [30 mM in H₂O] and assay buffer were premixed at 4° C. at a ratio of 100:30:10:145. 47.5 μl per well of this premix was transferred into polypropylene plates (MTP384, Nunc). Test compounds were dissolved and diluted in 100% DMSO and 2.5 μl added to the premix, then incubated at 37° C. for 3 h. At the end of the incubation period, 5 μl of the renin reaction (or standards in assay buffer) were transferred into EIA assays (as described above) and Ang I produced by renin was quantified. The percentage of renin inhibition (Ang I decrease) was calculated for each concentration of compound and the concentration of renin inhibition was determined that inhibited the enzyme activity by 50% (IC₅₀). The IC₅₀-values of all compounds tested are below 100 nM. However, selected compounds exhibit a very good bioavailability and are metabolically more stable than prior art compounds.

The compound of formula (I′) displays an IC₅₀-value of 0.5 nM.

### Hemodynamic Measurements (Telemetry Method)

Animals—Female double transgenic rats with human renin and human angiotensinogen were purchased from RCC Ltd, Füllingsdorf, Switzerland. All animals were maintained under identical conditions and had free access to normal pelleted rat chow and water. Rats were initially treated with enalapril (1 mg/kg/day) during 2 months. After approximately two weeks following cessation of enalapril treatment the double transgenic rats become hypertensive and reach mean arterial blood pressures in the range of 160-170 mmHg.

Transmitter implantation—The rats were anaesthetised with a mixture of 90 mg/kg Ketamin-HCl (Ketavet, Parke-Davis, Berlin FRG) and 10 mg/kg xylazin (Rompun, Bayer, Leverkusen, FRG) i.p. The pressure transmitter was implanted under aseptic conditions into the peritoneal cavity with the sensing catheter placed in the descending aorta below the renal arteries pointing upstream. The transmitter was sutured to the abdominal musculature and the skin closed.

Telemetry-System—Telemetry units were obtained from Data Sciences (St. Paul, Minn.). The implanted sensor consisted of a fluid-filled catheter (0.7 mm diameter, 8 cm long; model TA11PA-C40) connected to a highly stable low-conductance strain-gauge pressure transducer, which measured the absolute arterial pressure relative to a vacuum, and a radio-frequency transmitter. The tip of the catheter was filled with a viscous gel that prevents blood reflux and was coated with an antithrombogenic film to inhibit thrombus formation. The implants (length=2.5 5 cm, diameter=1.2 cm) weighted 9 g and have a typical battery life of 6 months. A receiver platform (RPC-1, Data Sciences) connected the radio signal to digitized input that was sent to a dedicated personal computer (Compaq, deskpro). Arterial pressures were calibrated by using an input from an ambient-pressure reference (APR-1, Data Sciences). Systolic, mean and diastolic blood pressure was expressed in millimeter of mercury (mmHg).

Hemodynamic measurements—Double transgenic rats with implanted pressure transmitters were dosed by oral gavage with vehicle or 10 mg/kg of the test substance (n=6 per group) and the mean arterial blood pressure was continuously monitored. The effect of the test substance is expressed as maximal decrease of mean arterial pressure (MAP) in the treated group versus the control group.

The compound of formula (I′) is active in this animal model. It leads to a blood pressure decrease of 26 mmHg at a single oral dose of 10 mg/kg, and a blood pressure decrease of 13 mmHg at a single oral dose of 3 mg/kg.

1. A compound of formula (I) named as (1R*,5S*)-7-{4-[3-(2-chloro-3,6-difluorophenoxy)propyl]phenyl}-3,9-diazabicyclo [3.3.1]non-6-ene-6-carboxylic acid cyclopropyl-(2,3-dichlorobenzyl)amide 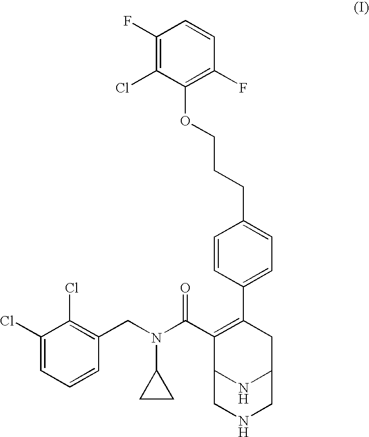
and optically pure enantiomers or a mixture of enantiomers such as a racemate; as well as pharmaceutically acceptable salts, solvent complexes and morphological forms thereof.

2. Enantiomer of the compound of formula (I) according to claim 1 represented by formula (I′) and named as (1R,5S)-7-{4-[3-(2-chloro-3,6-difluorophenoxy)propyl]phenyl}-3,9-diazabicyclo-[3.3.1]non-6-ene-6-carboxylic acid cyclopropyl-(2,3-dichlorobenzyl)amide 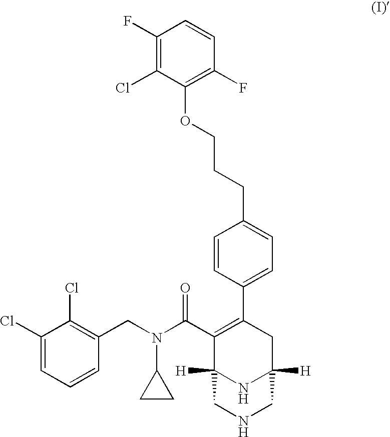
as well as pharmaceutically acceptable salts, solvent complexes and morphological forms thereof.

3. A pharmaceutical composition comprising a compound according to claim 1 and a pharmaceutically acceptable carrier material.

4. (canceled)

5. A method of treatment and/or prophylaxis of diseases selected from hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, glomerulonephritis, renal colic, complications resulting from diabetes such as nephropathy, vasculopathy and neuropathy, glaucoma, elevated intra-ocular pressure, atherosclerosis, restenosis post angioplasty, complications following vascular or cardiac surgery, erectile dysfunction, hyperaldosteronism, lung fibrosis, scleroderma, anxiety, cognitive disorders, complications of treatments with immunosuppressive agents, and other diseases known to be related to the renin-angiotensin system, comprising administering an effective amount of a compound according to claim 1 to a patient in need of such treatment or prophylaxis.

6. A compound according to claim 2 which is substantially free of other enantiomers of Formula I, in free or pharmaceutically acceptable salt form.

7. A pharmaceutical composition comprising a compound according to claim 2 and a pharmaceutically acceptable carrier material.

8. A method of treatment and/or prophylaxis of diseases selected from hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, glomerulonephritis, renal colic, complications resulting from diabetes such as nephropathy, vasculopathy and neuropathy, glaucoma, elevated intra-ocular pressure, atherosclerosis, restenosis post angioplasty, complications following vascular or cardiac surgery, erectile dysfunction, hyperaldosteronism, lung fibrosis, scleroderma, anxiety, cognitive disorders, complications of treatments with immunosuppressive agents, and other diseases known to be related to the renin-angiotensin system, comprising administering an effective amount of a compound according to claim 2 to a patient in need of such treatment or prophylaxis.

9. The method according to claim 8 wherein the disease is selected from hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, and complications resulting from diabetes such as nephropathy, vasculopathy and neuropathy.

10. A method of making a compound according to claim 1 comprising synthesizing a compound of formula I in N-protected form, deprotecting said N-protected compound, and recovering the compound of formula I.

11. A method according to claim 10 further comprising a chiral separation of the compound of formula I, in protected or unprotected form.

12. A method according to claim 10 comprising synthesizing a compound of formula I′ in N-protected form, deprotecting said N-protected compound, and recovering the compound of formula I′.
